(19) United States
(12) Patent Application Publication
(10) Pub. No.: US 2020/0179606 A1
(43) Pub. Date: Jun. 11, 2020

(54) DUAL-COMPARTMENT VITREOUS BODY INJECTOR

(71) Applicant: Trustees of Tufts College, Medford, MA (US)

(72) Inventors: Daniel Weinstein, Lincoln, MA (US); Daniel McGinn, Peabody, MA (US)

(21) Appl. No.: 16/631,599

(22) PCT Filed: Jul. 17, 2018
(86) PCT No.: PCT/US2018/042445

§ 371 Date: Jan. 16, 2020

Related U.S. Application Data
(60) Provisional application No. 62/533,819, filed on Jul. 18, 2017.

(51) Int. Cl. A61M 5/19; A61M 5/31; A61M 5/32; A61M 5/315

(52) U.S. Cl. CPC A61M 5/19; A61M 5/3148; A61M 5/3286; A61M 2005/3123; A61M 5/3297; A61M 2210/0612; A61M 5/31578

(57) ABSTRACT

A syringe for injection of fluid into the vitreous humor of an eye has a body that has, formed therein, an injection barrel and an extraction barrel, both of which have volumes that vary in response to movement of primary and secondary plungers that, are coupled to each other by a coupler. Two parallel needles, an extraction needle and an injection needle, emerge from the distal end of the body. Movement of the primary plunger injects fluid into the vitreous humor while at the same time causing the secondary plunger to extract the same volume of fluid from the vitreous humor, thus maintaining intraocular pressure.

### CROSS-REFERENCE TO RELATED APPLICATIONS

This application claims the benefit of U.S. Provisional Application No. 62/533,819, filed Jul. 18, 2017, all of which is incorporated herein by reference.

### FIELD OF INVENTION

This invention relates to injectors, and in particular, to injectors for injecting into the vitreous body of the eye.

### BACKGROUND

In some cases, it is necessary to inject a drug into the vitreous humor. This may be necessary in connection with treatment of such conditions as macular degeneration, myodesopsia, and retinal vein occlusions.

During an intra-vitreous drug injection, the additional fluid that enters the eye can increase intraocular pressure. This has certain side effects, ranging from discomfort to internal bleeding that may result in permanent eye damage.

To avoid such side effects, it is usual to inject only limited drug volumes. This limits the dose amount that can be given in one injection. As a result, a patient may have to return for repeated eyeball injections and may not receive multiple drugs in one injection. This is, to say the least, very unpleasant for the patient.

### SUMMARY

The invention features a dual-compartment injector that simultaneously delivers drug and removes vitreous fluid to maintain constant pressure in the eye. The device is operated the same way as a syringe; the surgeon pulls up on the plunger to withdraw drug solution into the injector and pushes down on a plunger to inject medicine. As it delivers medicine, the device simultaneously withdraws fluids from the eye through a second needle. This offers at least three advantages. First, the surgeon only needs one device to inject drug and withdraw fluid. And second, the device ensures that the exact amount of fluid is withdrawn at the proper time. And third, the device is compact and easily maneuverable.

In one aspect, the invention features an apparatus for injecting a fluid into vitreous humor of an eye. Such an apparatus includes a syringe having a body that has an injection barrel and an extraction barrel, both of which have corresponding first and second variable volumes. An injection needle and an extraction needle protrude from the distal end of the body. The injection needle connects to the injection barrel and the extraction needle connects to the extraction barrel. A coupler couples a primary and secondary plunger together so that the secondary plunger varies the second variable volume in respond to movement of the primary plunger.

In some embodiments, the coupler is a hydraulic coupler.

Embodiments include those in which the injection needle and extraction needle have beveled tips with the needles being oriented such that openings defined by the beveled tips face in different directions.

In other embodiments, the injection and extraction needles have distal openings that lies in corresponding first and second planes that are non-parallel.

In some embodiments, the extraction barrel and the injection barrel have the same cross-sectional areas.

In other embodiments, the extraction barrel and the injection barrel are conformal.

Among the embodiments are those in which the coupler comprises first and second arms connected by a connecting segment. In such embodiments, the primary plunger couples to the first arm and the secondary plunger couples to the second arm.

Also among the embodiments are those in which the coupler is configured to apply a first force to the secondary plunger in response to a second force exerted on the primary plunger, the first and second forces acting in opposite directions.

In some embodiments, the primary plunger comprises first and second rods having piston heads at distal ends thereof. The first rod passes the injection barrel and the second rod transmits a force to the secondary plunger via the coupler. Among these are embodiments in which the secondary plunger comprises third and fourth rods having piston heads at distal ends thereof. In these embodiments, the third rod passes through the extraction barrel and the fourth rod being receives a force from the primary plunger via the coupler.

Other embodiments include an extraction chamber that is in fluid communication with the extraction barrel and the extraction needle and an injection chamber that is in fluid communication with the injection barrel and the injection needle.

In some embodiments, the primary plunger comprises first and second rods that extend from the rod coupling toward a distal end of the body and a third rod that extends from the rod coupling away from the distal end of the body. The first piston-head is disposed at a distal end of the first rod and the second piston-head is disposed at a distal end of the second rod. The actuator is disposed at a distal end of the third rod.

Also among the embodiments are those in which the secondary plunger comprises a first rod, a second rod, a first piston-head, a second piston-head, and a rod coupling. In such embodiments, the first and second rod extend from the rod coupling toward a distal end of the body, the first piston-head is disposed at a distal end of the first rod, and the second piston-head is disposed at a distal end of the second rod.

Other embodiments include those in which a vent permits air to enter and leave the body in response to movement of the secondary plunger.

Yet other embodiments include something that cuts fibers. An example is a reciprocating hollow shaft through one of the needles. This shaft is configured to slice fibers that enter that needle. In such embodiments, one of the needles has an opening on a side thereof.

### BRIEF DESCRIPTION OF THE DRAWINGS

FIG. 1 shows a transverse view of an injector;

FIG. 2 shows an axial view of the injector of FIG. 1;

FIG. 3 shows an injector similar to that shown in FIG. 1 but with a cutting mechanism; and

FIG. 4 shows an injector having with coaxial injection and extraction needles.

### DETAILED DESCRIPTION

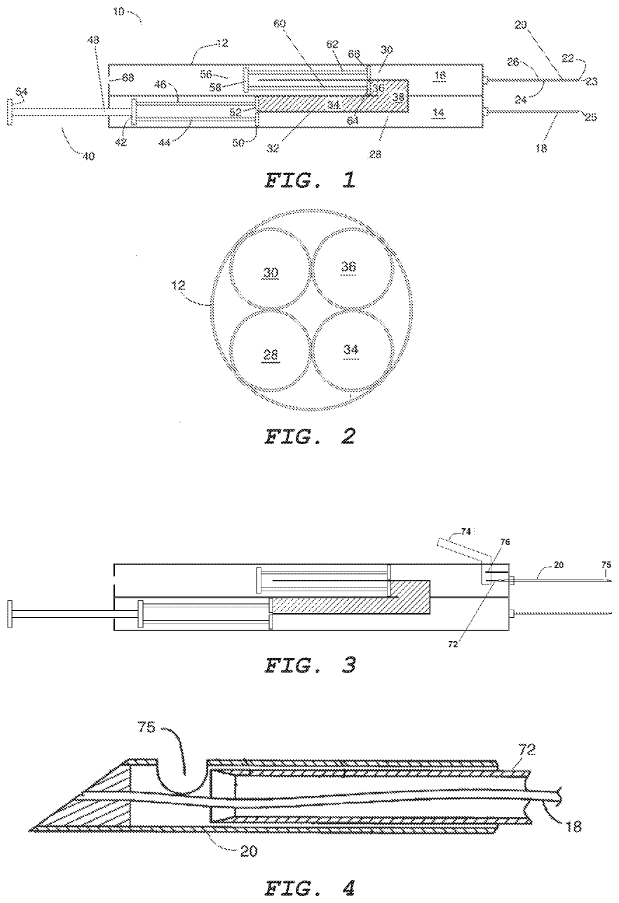

Referring to FIG. 1, an injector 10 includes a body 12 that extends along a longitudinal axis between proximal and distal ends thereof. An injection chamber 14 and an extraction chamber 16 occupy the distal end of the body 12.

A hollow injection needle 18 in fluid communication with the injection chamber 14 protrudes from the injection chamber's distal end. Similarly, a hollow extraction needle 20 in fluid communication with the extraction chamber 16 protrudes from the extraction chamber's distal end. The extraction needle 20 and the injection needle 18 both extend along the longitudinal direction.

In the illustrated embodiment, each needle 18, 20 has a beveled end 22. As a result of the beveled end 22, each needle 18, 20 has a length that varies with circumferential angle, as defined in a cylindrical coordinate system that has its axis coincident with the center of the needle. Because of the beveled end 22, each needle 18, 20 has a first side 24 at which its length is at a maximum and a second side 26 at which its length is at a minimum. As shown in the figure, the first side 24 of the injection needle 18 faces the first side 24 of the extraction needle 20. As a result, respective openings 23, 25 of the needles 18, 20 face in opposite directions.

Starting from the injection chamber 14, an injection barrel 28 extends along the body 12 in the proximal direction. Similarly, starting from the extraction chamber 16, an extraction barrel 30 extends along the body 12 in the proximal direction. The injection barrel 28 and the extraction barrel 30 have the same cross-sectional area. In a preferred embodiment, the injection barrel 28 and extraction barrel 30 are cylindrical barrels having the same radius.

Also formed in the body 12 is a hydraulic coupling 32. In the illustrated embodiment, the hydraulic coupling 32 is formed by first and second arms 34, 36 connected at distal ends thereof by a coupling segment 38. FIG. 2 shows a cross-section of the injector 10 shown in FIG. 1 taken along a plane proximal to the coupling segment 38 so that the injection and extraction barrels 28, 30 and the first and second arms 34, 36 of the hydraulic coupling 32 can be seen.

An axially movable primary plunger 40 protrudes from the proximal end of the barrel. The primary plunger 40 includes a first rod-coupling 42 that remains inside the body 12. First, second, and third rods 44, 46, 48 protrude axially outward from this first rod-coupling 42. The first and second rods 44, 46 extend axially in a distal direction and terminate at corresponding first and second piston-heads 50, 52.

The third rod 48 extends axially in the proximal direction, exits the body 12 altogether, and ends at its proximal end at an actuator 54. When a surgeon applies a force to the actuator 54, the third rod 48 transmits the force to the first rod-coupling 42, which then couples the force to the first and second rods 44, 46 and ultimately to the first and second piston-heads 50, 52. As a result, the primary plunger 40 moves axially as a single unitary structure in a direction consistent with the applied force.

The first piston-head 50 is shaped to conform to the cross-section of the injection barrel 28. As such, when the first piston-head 50 moves axially through the injection barrel 28, it changes the volume of the injection barrel 28.

The second piston-head 52 is disposed at a proximal end of the first arm 34 and shaped to conform to the cross-section of the first arm 34. As such, when the second piston-head 52 moves axially through the first arm 34, it changes the volume of the first arm 34.

Because of the first rod-coupling 42, the first piston-head 50 and the second piston-head 52 move together as a unit. Since the cross-sectional areas of the first arm 34 and the injection barrel 28 are the same, the change in the injection barrel's available volume corresponds to the change in volume in the first arm.

The injector 10 also includes a secondary plunger 56 that is identical to the primary plunger 40. However, this secondary plunger 56 is not actuated externally. Instead, the secondary plunger 56 is actuated internally by movement of the primary plunger 40.

As shown in FIG. 1, the secondary plunger 56 has a second rod-coupling 58 from which fourth and fifth rods 60, 62 extend toward the distal end of the body 12. The fourth rod 60 extends from the second rod-coupling 58 through the second arm 36 before terminating in a third piston-head 64. The fifth rod 62 extends from the second rod-coupling 58 through the extraction barrel 30 before terminating in a fourth piston-head 66.

The selection of ordinal numbers in the above description of the secondary plunger 56 is made to avoid conflict with similar components of the primary plunger 40. Accordingly, it is not intended to imply anything about the number of components of the secondary plunger 56. For example, the fact that the secondary plunger 56 has fourth and fifth rods 60, 62 is not intended to imply that it must three other rods.

As the third piston-head 64 moves axially through the second arm 36, it changes the volume of the second arm 36. As the fourth piston-head 66 moves axially through the extraction barrel 30, it changes the volume of the extraction barrel 30.

The third piston-head 64 and the fourth piston-head 66 move together as a unit. Since the cross-sectional areas of the second arm 36 and the extraction barrel 30 are the same, the change in the extraction barrel's volume tracks the change in the second arm's volume.

Operation begins with incompressible fluid filling the hydraulic coupling 32, the injection chamber 14 holding medicament to be injected though the injection needle 18, and the empty extraction chamber 16 standing ready to receive extracted fluid through the extraction needle 20.

The surgeon then penetrates the eyeball with both the injection needle 18 and the extraction needle 20 and then presses on the actuating surface 54. The resulting force simultaneously drives the first piston-head 50 and the second piston-head 52 distally. The first piston-head 50 forces the medicament out through the injection needle 18.

At the same time, the second piston-head 52 pushes distally against the incompressible medium. This transmits a force that pushes proximally against the third piston-head 64. The third rod 60 transmits this force to the second rod-coupling 58, which thus moves the second rod-coupling 58 proximally. This, in turn, applies a primal force to the fifth rod 62, which thus pulls the fourth piston-head 66 in the proximal direction.

Movement of the fourth piston-head 66 increases the volume of the extraction barrel 30, thus lowering pressure and therefore sucking fluid through the extraction needle 20 and into the extraction chamber 16. This increase in volume comes at the expense of a loss in volume of the space proximal to the fourth piston-head 66. A vent 68 in the body 12 ensures that there is no pressure increase in this space.

Because of the geometry described above, the volume of fluid flowing out through the injection needle 18 matches the volume of fluid sucked in through the extraction needle 20. As a result, there is no net change in the volume of fluid in the eyeball.

As discussed above, the needles 18, 20 are beveled and oriented so that the long sides of the needles face each other. The extraction point, located on the side of the extraction needle, faces away from the inject needle. As a result, the medicament is directed away from the extraction point 20 and is therefore unlikely to be extracted as it is being injected.

The vitreous body in an eye ball is composed of a gel-like matrix of interconnected collagen fibers with an optical density and viscosity slightly greater than that of water. The matrix is rooted to the side of the retina and provides anchorage points. To separate the fibers at the extraction point, and therefore mitigate the risk of a retinal tear, it is useful to embed a reciprocating cutter head 70 in the extraction needle 20, as shown in FIG. 3.

In one embodiment, the cutter head 58 is implemented as a hollow tube 72 having a sharpened distal edge that is fitted inside the extraction needle 20. A side opening 75 toward the distal end of the extraction needle 20 admits collagen fibers inside the extraction needle 20. In operation, The hollow tube 72 reciprocates so that its sharpened distal tip slides along the side opening 75 and thus severs whatever has penetrated into the opening.

At its opposite end, which lies inside the extraction chamber 16, the hollow tube 72 has a side opening that allows vitreous fluid to flow into the extraction chamber 16.

The hollow tube 72 is connected to a lever 74 inside the extraction tube. The lever 74 is fixed to the base of the injector 10 so that it can rotate about a bearing 76. Outside of the chambers, the lever 74 is driven by a solenoid switch. By varying the voltage to the solenoid switch, the lever 74 actuates the hollow tube 72 to move sinusoidally inside the extraction needle 20, separating the fibers from the vitreous fluid in much the same way that a guillotine separates a head from its associated body.

In FIGS. 1 and 3, the injection needle 18 and the extraction needle 20 are offset from each other.

In another embodiment, one needle may be inside the other. FIG. 4 illustrates one such embodiment in which the injection needle 18 is inside the extraction needle 20. In this embodiment, In this embodiment, the injection needle 18 is a tube that exits from the injection chamber 14 and enters the extraction needle 20. It then proceeds distally along the inside of the extraction needle 20. The openings through which fluid flow occurs are again facing in different directions. Vitreous humor enters through the side opening 75 and is suitably sliced by the moving hollow tube 72 on its way to the extraction chamber 16, whereas material stored in the injection chamber 14 exits through the distal end of the injection needle 18. In such cases, the injection needle 18 does not need to penetrate since the extraction needle 20 penetrates on behalf of the injection needle 18.

Having described the invention, and a preferred embodiment thereof, what is claimed as new, and secured by Letters Patent is:

1. An apparatus for injecting a fluid into vitreous humor of an eye, said apparatus comprising a syringe, said syringe comprising an injection needle, an extraction needle, a primary plunger, a secondary plunger, and a body, wherein said body has, formed therein, an injection barrel, an extraction barrel, and a coupler, wherein said injection barrel has a first variable volume, wherein said extraction barrel has a second variable volume, wherein said injection needle is in fluid communication with said injection barrel, wherein said extraction needle is in fluid communication with said extraction barrel, wherein said coupler couples said primary plunger to said secondary plunger, wherein said secondary plunger is configured to vary said second variable volume in response to movement of said primary plunger, and wherein said extraction needle and said injection needle protrude from a distal end of said body.

2. The apparatus of claim 1, wherein said injection needle and said extraction needle have beveled tips and wherein said needles are oriented such that openings defined by said beveled tips face in different directions.

3. The apparatus of claim 1, wherein said injection needle and said extraction needle are oriented such that openings thereof face in opposite directions.

4. The apparatus of claim 1, wherein said extraction barrel and said injection barrel have the same cross-sectional areas.

5. The apparatus of claim 1, wherein said extraction barrel and said injection barrel are conformal.

6. The apparatus of claim 1, wherein said coupler comprises first and second arms connected by a connecting segment and wherein said primary plunger couples to said first arm and wherein said secondary plunger couples to said second arm.

7. The apparatus of claim 1, wherein said coupler is configured to apply a first force to said secondary plunger in response to a second force exerted on said primary plunger, said first and second forces acting in opposite directions.

8. The apparatus of claim 1, wherein said primary plunger comprises first and second rods having piston heads at distal ends thereof, said first rod passing through said injection barrel and said second rod being configured to transmit a force to said secondary plunger via said coupler.

9. The apparatus of claim 1, further comprising first, second, third, and fourth rods, each of which has a piston head at a distal end thereof, wherein said first and second rods are constituents of said primary plunger and wherein said third and fourth rods are constituents of said secondary plunger, wherein said first rod passes through said injection barrel, wherein said second rod is configured to transmit a force to said secondary plunger via said coupler, wherein said third rod passes through said extraction barrel, and wherein said fourth rod is configured to receive a force from said primary plunger via said coupler.

10. The apparatus of claim 1, further comprising an extraction chamber and an injection chamber, wherein said extraction chamber is in fluid communication with said extraction barrel and said extraction needle and wherein said injection chamber is in fluid communication with said injection barrel and said injection needle.

11. The apparatus of claim 1, wherein said primary plunger comprises a first rod, a second rod, a first piston-head, a second piston-head, a third rod, an actuator, and a rod coupling, wherein said first and second rod extend from said rod coupling in a first direction, said first direction being toward a distal end of said body, wherein said third rod extends from said rod coupling in a second direction that is opposite said first direction, wherein said first piston-head is disposed at a distal end of said first rod, wherein said second piston-head is disposed at a distal end of said second rod, and wherein said actuator is disposed at a distal end of said third rod.

12. The apparatus of claim 1, wherein said secondary plunger comprises a first rod, a second rod, a first piston-head, a second piston-head, and a rod coupling, wherein said first and second rod extend from said rod coupling toward a distal end of said body, wherein said first piston-head is disposed at a distal end of said first rod, and wherein said second piston-head is disposed at a distal end of said second rod.

13. The apparatus of claim 1, wherein said body comprises a vent that permits air to enter and leave said body in response to movement of said secondary plunger.

14. The apparatus of claim 1, further comprising a hollow shaft that passes through said injection needle, wherein said hollow shaft is configured to slice fibers that enter said injection needle during reciprocation thereof.

15. The apparatus of claim 1, wherein said extraction needle has an opening on a side thereof.

16. The apparatus of claim 1, wherein said injection needle is disposed inside said extraction needle.

17. The apparatus of claim 1, wherein said injection needle and said extraction needle are offset from each other.

18. (canceled)

19. The apparatus of claim 1, wherein said injection needle has a distal opening that lies in a first plane, wherein said extraction needle has a distal opening that lies in a second plane, and wherein said first plane and said second plane are non-parallel.

20. The apparatus of claim 1, wherein at least one of said injection needles has an opening on a side thereof.

21. The apparatus of claim 1, further comprising a hollow shaft that passes through said extraction needle, wherein said hollow shaft, when reciprocating, slices through fibers that enter said extraction needle.
